(19) United States
(12) Patent Application Publication
(10) Pub. No.: US 2021/0341485 A1
(43) Pub. Date: Nov. 4, 2021

(54) Exosome Detection in Microfluidic Droplets

(71) Applicant: Northeastern University, Boston, MA (US)

(72) Inventors: Saheli SARKAR, Boston, MA (US); Tania KONRY, Boston, MA (US)

(21) Appl. No.: 17/266,671

(22) PCT Filed: Aug. 8, 2019
(86) PCT No.: PCT/US2019/045819

§ 371 Date: Feb. 8, 2021

Related U.S. Application Data
(60) Provisional application No. 62/716,033, filed on Aug. 8, 2018.

(51) Int. Cl. G01N 33/574; C12Q 1/6844; G01N 21/64

(52) U.S. Cl. CPC G01N 33/57488; C12Q 1/6844; G01N 2021/6439; G01N 21/6428; G01N 2458/10; G01N 21/6458

(57) ABSTRACT

A platform technology for the detection and quantification of biomarkers in exosomes secreted by single cells uses a microfluidic system and provides methods of diagnosis and prognosis of cancer and other conditions using the biomarkers. Single cells can be obtained from a subject and characterized by their exosome population. The biomarkers can be detected at extremely low levels using rolling circle amplification and multiplex analysis to enhance specificity and sensitivity of the analysis.

### BACKGROUND

Extracellular vesicles are secreted by cells and contain important biological cargo such as proteins, lipids, and genetic material. Cells use these vesicles to communicate with other cells, Researchers are attempting to leverage these vesicles to introduce drugs and therapeutic agents into cells. However, detecting cell-secreted extracellular vesicles, such as exosomes, which are generally only 30-100 nm in diameter and not themselves visible by light microscopy, is challenging with existing techniques.

### SUMMARY

The present technology provides a platform for the detection and quantification of biomarkers in exosomes secreted by single cells using a microfluidic system, as well as methods of diagnosis and prognosis of cancer and other conditions using the biomarkers. Single cells can be obtained from a subject and characterized by their exosome population. The biomarkers can be detected at extremely low levels using rolling circle amplification and multiplex analysis to enhance specificity and sensitivity of the analysis. The methods can be carried out rapidly and inexpensively compared to other methods of exosome analysis requiring biochemical isolation or large amounts of cells or body fluids.

The present technology can be further summarized by the following aspects:

1. A method of detecting and/or quantifying a biomarker secreted from single cells, the method comprising the steps of:

(a) providing a microfluidic system configured to encapsulate single cells and one or more reagents into microfluidic aqueous droplets in oil, and to perform fluorescence imaging microscopy of the droplets;

(b) encapsulating single cells combined with one or more detection reagents for said biomarker in microfluidic droplets of the system provided in (a); and

(c) detecting and/or quantifying the biomarker in extracellular spaces of the microfluidic droplets.

2. The method of aspect 1, wherein the biomarker is associated with an exosome, and the exosome is detected or quantified.
3. The method of aspect 1 or 2, wherein step (c) comprises performing a rolling circle amplification (RCA) reaction in the microfluidic droplets.
4. The method of any of the previous aspects, wherein the biomarker is a nucleic acid or a protein.
5. The method of aspect 3, wherein the biomarker is a nucleic acid and the RCA reaction amplifies the nucleic acid.
6. The method of aspect 3, wherein the biomarker is a protein, wherein one of the one or more reagents is a binding agent that specifically binds to the protein, and wherein the binding agent is linked to a nucleic acid tag or bar code which is amplified in the RCA reaction.
7. The method of any of the preceding aspects, wherein the biomarker is selected from the group consisting of CD63, CD9, CD81, ALIX, perforin, FasL and EGFR.
8. The method of any of the preceding aspects, wherein two or more biomarkers are detected and/or quantified.
9. The method of any of the preceding aspects, wherein the cells are obtained from a subject.
10. The method of aspect 9, wherein the subject is a human or other mammal.

11. The method of aspect 9 or 10, wherein the method is performed to aid in diagnosis or prognosis of a medical condition of the subject.

12. The method of aspect 11, wherein the medical condition is cancer.
13. A kit comprising (i) a microfluidic device configured to encapsulate single cells and one or more reagents into microfluidic aqueous droplets in oil, (ii) one or more reagents capable of detecting and/or quantifying a biomarker secreted from a cell, and optionally (iii) one or more reagents for performing an RCA reaction.
14. The kit of aspect 13, wherein the biomarker is associated with an exosome.
15. The kit of aspect 13 or 14, wherein the kit comprises reagents for the detection and/or quantification of two or more biomarkers secreted from a cell.

### DETAILED DESCRIPTION

The present technology utilizes microfluidic devices that entrap single living cells or groups of single cells in microfluidic droplets, and detects within the droplets, and optionally quantifies, molecules secreted in the form of exosomes from those cells by isothermal rolling circle amplification (RCA). The technology employs multiplex detection of cell-specific markers for the detection of tumor cells and/or the diagnosis or prognosis of disease by examining cells obtained from a subject. Target molecules for detection and/or quantification include proteins, glycoproteins, peptides, nucleic acid molecules (RNA or DNA), and lipids, as well as metabolites and other small molecules found in exosomes.

The technology described herein can be employed in a commercial format, such as a large scale, automated, or screening format, to detect exosomes from clinical samples from patients and from various cell types. The technology also can be used in academic or industrial labs as well as hospitals. Exosomes contain biomarkers that can be used for the diagnosis or prognosis of diseases or medical conditions, and such diagnosis or prognosis can be combined with treatment or intervention to treat or prevent disease or the progression of a disease or medical condition.

Some existing techniques for exosome detection involve specialized instruments (e.g., a qNano particle size analyzer by Izon Science Ltd, which uses tunable resistive pulse sensing (TRPS)) or highly expensive and complex sensors (e.g., nano-plasmonic sensors). However, the present technology uses standard microfluidic methods, commonly-used polydimethylsiloxane-based devices, and easily available materials and equipment. Another existing methodology for detecting exosomes is the enzyme-linked immunosorbent assay (ELISA), which requires a large quantity of exosomes. The present methodology can detect and identify or categorize single exosomes in microfluidic droplets, thereby reducing both the required amount of biological material and reagent consumption. Existing procedures also require isolating exosomes by time-consuming and complicated processes such as ultracentrifugation or liquid chromatography before detection. In contrast, the present technology is designed to detect exosomes secreted by cells directly, without prior isolation steps; it is therefore faster, cheaper, and far more sensitive.

Exosomes are small membrane-bound extracellular vesicles that are secreted from eukaryotic cells. Exosomes originate in the endosomal compartment and form within multivesicular bodies, which later fuse with the plasma membrane and release the exosomes from the cell. Exosomes are thought to play a role in intercellular communication, development, immune responses, and disease. Exosomes derived from tumor cells carry tumor antigens and stimulate the anti-tumor immune response, but also can stimulate tumor cell growth and migration. Given their small size of exosomes (e.g., less than 100 nm, less than 300 nm, or less than 500 nm, or about 300-500 nm or about 100-500 nm) they can be isolated by ultracentrifugation or size exclusion chromatography. They also can be isolated by immunoadsorption, such as to magnetic beads coated with antibodies that bind phosphatidylserine (enriched in exosomes) or CD63 protein. For further background and methodology related to exosomes, see Li et al., J. Hematol. & Oncol. (2017) 10:175, hereby incorporated by reference.

Exosomes secreted by specific cell types contain molecules such as proteins, lipids, metabolites, and RNA (e.g., mRNA, miRNA), which can be used as cell specific markers. Exosomes also contain molecules that are common to most extracellular vesicles and therefore can be used to detect the presence of exosomes themselves in an extracellular fluid. Using the present technology, exosomes secreted from individual cells can be detected and screened by detecting and/or quantifying a selected panel of markers and using multiplexed detection of two or more such markers to specifically identify exosomes from a cell population of interest. Any known biomarker for exosomes in general, such as CD63, CD9, or CD81, can be used, as well as any known cell-specific exosome markers, such as CD63, ALIX, perforin, and FasL (found in exosomes from NK cells), glyptican-1 (GPC1) (found in cancer cells), or EGFR (found in gastric cancer cells). For further exosome biomarkers known to be produced by cancer cells see Huang et al., Int. J. Biol. Sci. Sci. (2019) 15(1):1-11.

Exosomes cannot be detected by light microscopy due to their small size, and therefore cannot be detected directly in a microfluidic system. Nevertheless, the rapid mixing and kinetics associated with aqueous microdroplets formed in an oil stream in microfluidic devices enables rapid detection of biomolecules secreted by cells. Individual cells can be readily encapsulated singly or as two or more cells, either associated or not associated, with detection reagents in individual microdroplets. This makes possible the detection of any material secreted by one or more cells within a droplet, such as extracellular vesicles including exosomes and their molecular constituents. This makes possible the identification of the origin of the detected molecules or combination of molecules with respect to individual cells within the microdroplet. The present technology also makes possible the detection of molecules and vesicles secreted by individual cells within a population, The population of cells can be distributed within a plurality of microdroplets, with one or more cells per droplet, or one cell or less per droplet, thus providing information on the heterogeneity of single cell responses and on combinations of molecules present in exosomes from individual cells or types of cells.

The present technology provides methods for detecting and/or quantifying exosomes secreted by single cells or groups of cells (e.g., groups of 2, 3, 4, 5, 6, 7, 8, 9, or 10 cells, or 2-3 cells, 2-4 cells, 2-5 cells, or 2-10 cells), either constitutively released or released in response to a stimulus. The cells can be cells obtained from a subject, such as a human or other mammal, or cells of a pathogen. The cells can be normal cells, cancer cells, cells of the immune system (e.g., dendritic cells, macrophages, B cells, T cells, NK cells). While exosomes are generally detected in an extracellular space, following their secretion by a cell, they also may be detected within a cell, either prior to secretion or after uptake by a second cell after secretion by a first cell. The presence, number, or content of exosomes secreted by a cell, or the time course of their secretion following a stimulus to the cell, can be used to diagnose the presence or extent of a disease or to predict the outcome of a disease or the effectiveness of a chosen therapy, or to provide information with regard to intercellular communication.

In order to detect exosomes or other material secreted by single cells, a suspension of the cells in an aqueous buffer or cell culture medium is loaded into a microfluidic device and mixed with suitable reagents upon entry, and the cell/reagent mixture is then formed into a sequence of microfluidic aqueous droplets suspended in a flowing stream of oil. Methods for obtaining single cells, either of a single type or containing a mixture of different cell types, are known. For example, individual cells can be obtained from blood or another body fluid, or can be obtained by mechanically disrupting and/or enzymatically dissociating a tissue sample. Preferably living cells are used. Devices and methods for producing suitable microdroplets, storing them in arrays, and observing and imaging the microdroplets by fluorescence microscopy are known in the art, and described, for example, in WO2017/011819A1. Methods are also known for harvesting individual cells following analysis of their released exosomes, so that the analyzed individual cells can have their genome, transcriptome, or proteome analyzed, or so that they can be cultured or stored for further study.

Detection and characterization of exosomes in a microfluidic system can be tested or calibrated using isolated exosomes in the place of a feed source containing a cell suspension. Any method known for isolating and purifying exosomes from a cell source, such as a cell culture or tissue, or a body fluid, can be used. See, e.g., Huang et al., Int. J. Biol. Sci. Sci. (2019) 15(1):1-11; see also www.labome.com/method/Exosomes-Isolation-and-Characterization-Methods-and-Specific-Markers.html.

Rolling circle amplification (RCA) is preferred in the present technology as a highly sensitive and specific technique for detecting biomarkers located on, within, or derived from exosomes. RCA is a highly specific technique which can be used to detect and quantify biomolecules present at very low levels, because it can generate amplicon product with a high intensity of fluorescence. RCA also can provide multiplex detection of several markers in exosomes, thereby allowing different types or subsets of exosomes secreted by the same cell or different cells to be detected simultaneously. In one embodiment, the use of RCA allows the recognition of multiple targets on exosomes by hybridizing oligonucleotides either to mRNA or miRNA that is associated with or released from the exosomes themselves. In another embodiment, RCE is used to detect oligonucleotide tags or barcodes that are specifically coupled to antibodies and used to detect protein markers associated with or released from exosomes.

Fluorescence-based RCA can be used for detection of exosome biomarkers to obtain qualitative and/or quantitative information on cell-specific exosome secretion. The RCA assay yields amplicons which correspond to an individual target molecule, and is capable of detecting a single DNA amplicon molecule per cell. See Konry et al. (2010) Small 7(3):395. Either the amplicons themselves can be fluorescent, due to the inclusion of one or more fluorescently tagged nucleoside triphosphates in the microdroplets, or two or more amplicons can be detected and distinguished within single droplets by the use of probes, such as molecular beacons, with probes specific for each amplicon sequence designed to fluoresce at a unique wavelength range upon binding to their respective amplicon. Compared to other types of exosome detection assays, conduction of RCA in a droplet format reduces reagent consumption, accelerates the detection process, and identifies exosomes secreted by single cells. In order to perform RCA to detect exosome biomarkers using the present technology, single cells can be co-encapsulated with oligonucleotide probes and an RCA reaction mixture together in droplets. Exosome secretion can be detected and their level of secretion compared between various stimulant conditions.

RCA can be performed in microfluidic droplets, for example, by the inclusion of the following reagents in a mixture that is combined with cells to form the microdroplets: i) a ligase to circularize a linear DNA or RNA tag or barcode, or mRNA or miRNA to be detected (alternatively, a generic DNA circle can be formed by ligating the tag/barcode or RNA for detection and a circle-forming DNA segment, e.g., by coupling a biotinylated antibody to a biotinylated circle-forming DNA molecule using an avidin bridge); ii) one or more amplification primers that specifically bind to the ligated, circularized DNA or RNA; iii) a polymerase that amplifies the circular DNA, forming a concatamer containing multiple copies of the circular DNA; and iv) a molecular beacon or other fluorescent probe that provides a fluorescence signal when bound to the amplified circular DNA. A polymerase is used that can perform the amplification step at room temperature or another suitable temperature, such as 37C. See Konry et al. (2010) Small 7(3):395.

In a multiplex format, two or more molecular beacons can be used which provide differently colored fluorescence emissions when their respective target sequences are bound. Certain types of probes can combine fluorophores having different emission wavelengths, so that multiple different molecular targets can be detected simultaneously using RCA and suitable fluorescence microscopy equipment. Thus, a single multiplex RCA reaction in an aqueous microdroplet can simultaneously detect and/or quantify 2 or more, 3 or more, 4 or more, 5 or more, 6 or more, 7 or more, 8 or more, 10 or more, 12 or more, 16 or more, 20 or more, 30 or more, 50 or more, or 100 or more different molecular targets. Alternatively, a multiplex analysis can be carried out serially, i.e., by performing a first RCA reaction using a first targeted DNA or RNA tag or barcode, detecting and/or quantifying the results, then merging, using a droplet merging junction, the analyzed microdroplets with another microdroplet containing a second, differently targeted DNA or RNA tag or barcode and performing a second RCA reaction, detecting and/or quantifying the results, and optionally repeating the droplet merging and reacting with third, optionally fourth, and optionally further droplets containing third, fourth, and further differently targeted DNA or RNA tags or barcodes to detect and/or analyze third, fourth, and further exosome or other biomarkers. The combined detection and/or quantification of combinations of 2, 3, 4, or more biomarkers per cell produces information about the source or type of cell, its physiological or pathological state, its gene expression or genetic makeup, its ability to communicate with other cells, or other aspects of exosome function.

In addition to detecting and quantifying biomarkers and combinations of biomarkers present in exosomes of different cell types, or associated with different cell physiological or pathological states, the technology described herein can be used to assess changes in exosome composition, secretion, or effects on other cells by designing suitable assays that can be performed using an aqueous microdroplet-microfluidic system. In one embodiment, a stimulus such as a cytokine, hormone, immunomodulatory agent, another cell, or an antibody or other agent known or suspected of altering the analyzed cell's physiology, state of activation, metabolism, or survival can be introduced, either when initially forming the microdroplet containing the analyzed cell, or by merging the micrdroplet containing the analyzed cell with another microdroplet containing a test agent or stimulus at a droplet merging junction. After introducing a test agent, the microdroplets containing analyzed cells can be transported to an array of microdroplet chambers or docking stations for incubation and subsequent analysis of exosome biomarkers, such as by RCA or multiplex RCA.

What is claimed is:

1. A method of detecting and/or quantifying a biomarker secreted from single cells, the method comprising the steps of:
(a) providing a microfluidic system configured to encapsulate single cells and one or more reagents into microfluidic aqueous droplets in oil, and to perform fluorescence imaging microscopy of the droplets;
(b) encapsulating single cells combined with one or more detection reagents for said biomarker in microfluidic droplets of the system provided in (a); and
(c) detecting and/or quantifying the biomarker in extracellular spaces of the microfluidic droplets.

2. The method of claim 1, wherein the biomarker is associated with an exosome, and the exosome is detected or quantified.

3. The method of claim 1, wherein step (c) comprises performing a rolling circle amplification (RCA) reaction in the microfluidic droplets.

4. The method of claim 1, wherein the biomarker is a nucleic acid or a protein.

5. The method of claim 3, wherein the biomarker is a nucleic acid and the RCA reaction amplifies the nucleic acid.

6. The method of claim 3, wherein the biomarker is a protein, wherein one of the one or more reagents is a binding agent that specifically binds to the protein, and wherein the binding agent is linked to a nucleic acid tag or bar code which is amplified in the RCA reaction.

7. The method of claim 1, wherein the biomarker is selected from the group consisting of CD63, CD9, CD81, ALIX, perforin, FasL and EGFR.

8. The method of claim 1, wherein two or more biomarkers are detected and/or quantified.

9. The method of claim 1, wherein the cells are obtained from a subject.

10. The method of claim 9, wherein the subject is a human or other mammal.

11. The method of claim 9, wherein the method is performed to aid in diagnosis or prognosis of a medical condition of the subject.

12. The method of claim 11, wherein the medical condition is cancer.

13. A kit comprising (i) a microfluidic device configured to encapsulate single cells and one or more reagents into microfluidic aqueous droplets in oil, (ii) one or more reagents capable of detecting and/or quantifying a biomarker secreted from a cell, and optionally (iii) one or more reagents for performing an RCA reaction.

14. The kit of claim 13, wherein the biomarker is associated with an exosome.

15. The kit of claim 13, wherein the kit comprises reagents for the detection and/or quantification of two or more biomarkers secreted from a cell.
